(19) United States
(12) Patent Application Publication
(10) Pub. No.: US 2017/0326355 A1
(43) Pub. Date: Nov. 16, 2017

(54) IMPLANTABLE MEDICAL DEVICE FOR VASCULAR DEPLOYMENT

(71) Applicant: CARDIAC PACEMAKERS, INC., St. Paul, MN (US)

(72) Inventors: Brendan Early Koop, Ham Lake, MN (US); Angelo Auricchio, Magdeburg (DE); Benjamin J. Haasl, Forest Lake, MN (US); Brandon Christopher Fellows, Chicago, IL (US)

(73) Assignee: CARDIAC PACEMAKERS, INC., St. Paul, MN (US)

(21) Appl. No.: 15/590,811

(22) Filed: May 9, 2017

Related U.S. Application Data
(60) Provisional application No. 62/334,156, filed on May 10, 2016.

(51) Int. Cl. A61N 1/05; A61B 5/0215; A61B 5/00; A61N 1/365; A61N 1/368; A61N 1/362; A61B 5/042; A61M 25/04

(52) U.S. Cl. CPC A61N 1/059; A61N 1/3686; A61N 1/05; A61B 5/0215; A61B 5/686; A61N 1/3621; A61N 1/36514; A61B 5/0031; A61B 5/042; A61M 25/04; A61N 1/36564

(57) ABSTRACT

A leadless cardiac pacemaker (LCP) may be deployed within a patient's vasculature at a location near the patient's heart in order to pace the patient's heart and/or to sense electrical activity within the patient's heart. In some cases, an LCP may be implanted within the patient's superior vena cava or inferior vena cava. The LCP may include an expandable anchoring mechanism configured to secure the LCP in place.

### CROSS REFERENCE TO RELATED APPLICATIONS

This application claims the benefit of U.S. Provisional Patent Application Ser. No. 62/334,156, filed on May 10, 2016, the disclosure of which is incorporated herein by reference.

### TECHNICAL FIELD

The disclosure relates generally to implantable medical devices, and more particularly relates to implantable medical devices that can be deployed within the vasculature near the patient's heart.

### BACKGROUND

Implantable medical devices are commonly used today to monitor a patient and/or deliver therapy to a patient. For example, implantable sensors are often used to monitor one or more physiological parameters of a patient, such as heart beats, heart sounds, ECG, respiration, etc. In some instances, pacing devices are used to treat patients suffering from various heart conditions that may result in a reduced ability of the heart to deliver sufficient amounts of blood to a patient's body. Such heart conditions may lead to slow, rapid, irregular, and/or inefficient heart contractions. To help alleviate some of these conditions, various medical devices (e.g., pacemakers, defibrillators, etc.) can be implanted in a patient's body. Such devices may monitor and in some cases provide electrical stimulation to the heart to help the heart operate in a more normal, efficient and/or safe manner.

### SUMMARY

This disclosure provides design, delivery and deployment methods, and clinical usage alternatives for medical devices. In one example, the disclosure is directed to implantable medical devices that may be configured to be disposed within the vasculature near a patient's heart in order to pace a portion of the patient's heart and/or to sense electrical activity within the patient's heart. In some cases, an implantable medical device may be implantable within the vasculature near the right atrium of the patient's heart, and may be configured to pace the right atrium of the patient's heart and/or sense cardiac signals in the right atrium of the patient's heart.

In an example of the disclosure, a leadless cardiac pacemaker (LCP) may be configured for deployment within a patient's vasculature at a location near the patient's heart. In some cases, the LCP may include an elongated housing that has opposing ends, and a side wall extending between the opposing ends. The elongated housing may have a length dimension between the opposing ends and a width dimension normal to the length dimension. The length dimension may be larger than the width dimension, and sometimes substantially larger. A power source may be disposed within the elongated housing. Circuitry disposed within the elongated housing may be operatively coupled to the power source and may be configured to pace the patient's heart and/or sense electrical activity of the patient's heart. An anode electrode and a cathode electrode may each be operatively coupled to the circuitry and may each be fixed relative to the elongated housing. The cathode electrode may be spaced from the anode electrode and may be positioned along the side wall of the elongated housing. The cathode electrode may have a surface area that is smaller than a surface area of the anode electrode, and in some cases substantially smaller. In some cases, an expandable anchoring mechanism may be secured to the elongated housing. The expandable anchoring mechanism may have a collapsed configuration for delivery and an expanded configuration that locates the LCP within the patient's vasculature, with the cathode electrode in engagement with the patient's vasculature.

Alternatively or additionally, the expandable anchoring mechanism is configured to anchor the LCP in the patient's vasculature such that the length dimension of the elongated housing is positioned substantially parallel with blood flow in the patient's vasculature.

Alternatively or additionally to any of the embodiments above, the anode electrode is disposed proximate a first opposing end of the elongated housing.

Alternatively or additionally to any of the embodiments above, the cathode electrode is disposed proximate a second opposing end of the elongated housing.

Alternatively or additionally to any of the embodiments above, the LCP further includes a retrieval feature disposed proximate at least one of the opposing ends of the elongated housing.

Alternatively or additionally to any of the embodiments above, the expandable anchoring mechanism includes a side wall defining an inner surface and an outer surface. The elongated housing is secured to the inner surface of the expandable anchoring mechanism with the cathode electrode extending laterally outwardly from the elongated housing in the width dimension and through the side wall to engage the patient's vasculature. Alternatively, the elongated housing is secured to the outer surface of the expandable anchoring mechanism with the cathode electrode held in engagement with the patient's vasculature.

Alternatively or additionally to any of the embodiments above, the LCP further includes a lead structure extending from the elongated housing, the lead structure including at least one additional electrode that is operatively coupled to the circuitry and configured to extend into the patient's heart from the patient's vasculature at the location near the patient's heart.

Alternatively or additionally to any of the embodiments above, the expandable anchoring mechanism is configured to anchor the LCP in the patient's superior vena cava proximate the patient's right atrium. Alternatively, the expandable anchoring mechanism is configured to anchor the LCP in the patient's inferior vena cava proximate the patient's right atrium.

Alternatively or additionally to any of the embodiments above, the LCP further includes a tether woven into an end of the expandable anchoring mechanism, wherein pulling on the tether enables the expandable anchoring mechanism to be at least partially collapsed from its expanded configuration for repositioning of the LCP.

Alternatively or additionally to any of the embodiments above, the expandable anchoring mechanism is configured such that in its expanded configuration, the expandable anchoring mechanism exerts sufficient outward force on the patient's vasculature to secure the LCP in place with the cathode electrode in engagement with the patient's vasculature.

Alternatively or additionally to any of the embodiments above, the expandable anchoring mechanism includes a side wall defining an inner surface and an outer surface, and wherein the expandable anchoring mechanism includes fixation tines that extend outwardly from the outer surface of the side wall.

Alternatively or additionally to any of the embodiments above, the expandable anchoring mechanism has a length in its expanded configuration that is less than the length dimension of the elongated housing.

In another example of the disclosure, an implantable medical device (IMD) is configured for deployment within a patient's vena cava, proximate the patient's right atrium, in order to pace the right atrium and/or sense electrical activity within the right atrium. The IMD may include a housing that is configured to be positioned within the patient's vena cava, the housing having a first end and an opposing second end with a side wall extending between the first end and the opposing second end. A power source and circuitry are disposed within the housing and the circuitry is operably coupled to the power source. An anode electrode may be positioned proximate the first end of the housing and a cathode electrode may be spaced from the first end of the housing. The cathode electrode has a surface area that is smaller than a surface area of the anode electrode, and sometimes substantially smaller. The anode electrode and the cathode electrode may be operatively coupled to the circuitry. An expandable anchoring mechanism may be secured to the housing. The expandable anchoring mechanism may have a collapsed configuration for delivery and an expanded configuration that locates the IMD within the vena cava, with the cathode electrode in engagement with the vena cava.

Alternatively or additionally, the IMD further includes a retrieval feature disposed proximate the first end of the housing.

Alternatively or additionally to any of the embodiments above, the IMD further includes a lead having at least one additional electrode that is operatively coupled to the circuitry, the lead is configured to extend into the patient's heart.

Alternatively or additionally to any of the embodiments above, the lead is configured to bias the at least one additional electrode against a wall of the right atrium.

In another example of the disclosure, a leadless cardiac pacemaker (LCP) is configured for deployment within a patient's vasculature at a location near the patient's heart. The LCP includes a housing that is configured to be positioned within the patient's vasculature proximate the patient's heart. The LCP includes a power source disposed within the housing. Circuitry may be disposed within the housing and may be operatively coupled to the power source. A lead is configured to extend into the patient's heart and includes at least one electrode that is operatively coupled to the circuitry. The circuitry is configured to pace the patient's heart and/or sense electrical activity of the patient's heart using at least one electrode of the lead. An expandable anchoring mechanism may be coupled to the LCP. The expandable anchoring mechanism may have a delivery configuration and an anchoring configuration, where the anchoring configuration anchors the LCP within the patient's vasculature at the location near the patient's heart.

Alternatively or additionally, the expandable anchoring mechanism is configured to anchor the LCP within the superior vena cava or the inferior vena cava, and the lead is configured to bias the at least one electrode against a wall of the right atrium of the patient's heart.

The above summary of some illustrative embodiments is not intended to describe each disclosed embodiment or every implementation of the present disclosure. The Figures and Description which follow more particularly exemplify these and other illustrative embodiments.

### BRIEF DESCRIPTION OF THE FIGURES

The disclosure may be more completely understood in consideration of the following description in connection with the accompanying drawings, in which:

FIG. 1 is a schematic illustration of a human heart;

FIG. 2 is a schematic illustration of an implantable medical device (IMD) configured to be implanted within the vasculature near the heart;

FIG. 3 is a schematic diagram of an illustrative IMD;

FIG. 4 is a schematic diagram of an illustrative IMD;

FIG. 5 is a schematic diagram of an illustrative IMD;

FIG. 6 is a schematic diagram of an illustrative IMD including a tether for possible repositioning and/or removal of the IMD;

FIG. 7 is a schematic diagram of an illustrative IMD prepared for delivery;

FIGS. 8A and 8B are schematic illustrations of an IMD with the expandable anchoring mechanism in a collapsed configuration and then in an expanded configuration;

FIGS. 9A and 9B are schematic illustrations of an IMD with the expandable anchoring mechanism in a collapsed configuration and then in an expanded configuration;

FIG. 10 is a schematic illustration of an IMD prepared for delivery;

FIG. 11 is a schematic illustration of an IMD implanted within the superior vena cava, with a lead structure extending into the right atrium; and

FIG. 12 is a schematic block diagram of an illustrative leadless cardiac pacemaker (LCP), which may be considered as being an example housing in one of the IMDs of FIGS. 2 through 11.

While the disclosure is amenable to various modifications and alternative forms, specifics thereof have been shown by way of example in the drawings and will be described in detail. It should be understood, however, that the intention is not to limit the disclosure to the particular embodiments described. On the contrary, the intention is to cover all modifications, equivalents, and alternatives falling within the spirit and scope of the disclosure.

### DESCRIPTION

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used connection with other embodiments whether or not explicitly described unless clearly stated to the contrary.

The following description should be read with reference to the drawings in which similar structures in different drawings are numbered the same. The drawings, which are not necessarily to scale, depict illustrative embodiments and are not intended to limit the scope of the disclosure.

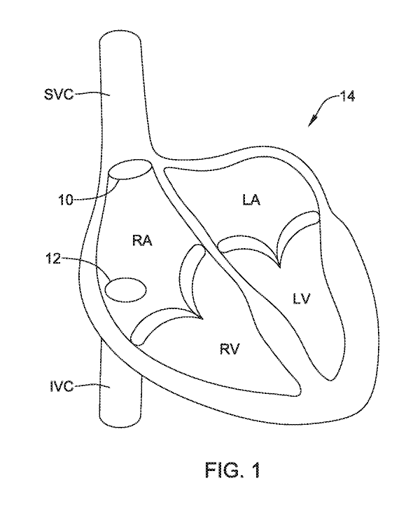

FIG. 1 is a schematic illustration of a heart H, illustrating a right atrium RA, a right ventricle RV, a left atrium LA and a left ventricle LV. For simplicity, some of the vasculature around the heart H, such as the aorta, the pulmonary arteries and the pulmonary veins are not shown. However, the superior vena cava (SVC), which returns blood from the upper body to the right atrium RA, and the inferior vena cava (IVC), which returns blood from the lower body to the right atrium RA are shown. The SVC extends to an SVC terminus 10, where the SVC is fluidly coupled with the right atrium RA. The IVC extends to an IVC terminus 12, where the IVC is fluidly coupled with the right atrium RA. In some cases, an implantable medical device (IMD) may be implanted within the SVC or the IVC such that the IMD may be able to sense electrical cardiac activity within the right atrium RA from within the SVC or IVC. In some instances, as will be discussed, an IMD disposed within the SVC or the IVC may include a lead structure that extends into the heart H, such as into the right atrium RA.

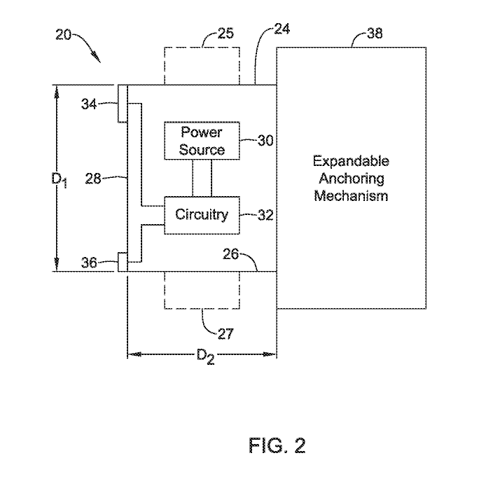

FIG. 2 is a schematic diagram of an illustrative IMD 20 that may, for example, be implantable within the vena cava, such as the SVC or the IVC. The illustrative IMD 20 includes a housing 22. In some cases, the housing 22 includes opposing ends 24 and 26, with a side wall 28 extending between the opposing ends 24 and 26. The housing 22 may, for example, be considered as being an elongated housing, having a length dimension denoted by a dimension D1 and a width dimension that is normal to the length direction and that is denoted by a dimension D2. In some cases, D1 is larger than D2. In some instances, D1 is at least twice D2, or at least three times D2, or in some cases D1 is at least four times D2. A power source 30 may be disposed within the housing 22. In some cases, the power source 30 may be a battery. In some cases, the power source 30 may be rechargeable, such as a rechargeable battery, a capacitor such as a super-capacitor and/or any other suitable rechargeable power source. Circuitry 32 is disposed within the housing 22 and may be operably coupled to the power source 30. In some cases, the circuitry 32 may be configured to sense the heart H and/or to sense electrical activity of the heart H.

In the example shown, an anode electrode 34 is fixed relative to the housing 22. In some cases, a cathode electrode 36 may be fixed relative to the housing 22 and may be spaced apart from the anode electrode 34. In some cases, the anode electrode 34 may be disposed proximate the first end 24 of the housing 22 while the cathode electrode 36 may be disposed proximate the second end 24 of the housing 22, but this is not required in all cases. In some cases, the cathode electrode 36 may be positioned along the side wall 28. In some cases, the cathode electrode 36 may extend radially outwardly from the side wall 28 to facilitate good engagement between the cathode electrode 36 and surrounding tissue. The anode electrode 34 may also be on the side wall 28, or may be at an end 24 or located elsewhere. The anode electrode 34 and the cathode electrode 36 may each be operatively coupled to the circuitry 32. In some cases, the cathode electrode 36 may be considered as having a surface area that is smaller than a surface area of the anode electrode 34. In some cases, the anode electrode 34 may have a surface area that is at least twice that of the cathode electrode 36, at least three times, at least four times, or at least ten times, that of the cathode electrode 36.

In some cases, the housing 22 may include one or more retrieval features, such as a retrieval feature 25 that is located at or near the first end 24 and/or a retrieval feature 27 that is located at or near the second end 26. The housing 22 may include no retrieval features, one retrieval feature, two retrieval features, or more than two retrieval features. The retrieval features 25 and 27, if present, may take any desired shape or configuration. In some cases, the retrieval features 25 and 27, if present, may take the form of a knob, clasp, hook or other feature that can be engaged by a snare or other retrieval device, for example.

The illustrative IMD 20 also includes an expandable anchoring mechanism 38 that is secured to the housing 22. In some cases, the housing 22 may be disposed within the expandable anchoring mechanism 38. In some cases, the housing 22 may be secured to an outside of the expandable anchoring mechanism 38. The expandable anchoring mechanism 38 may, for example, have a collapsed or delivery configuration to facilitate delivery through the vasculature to a location such as but not limited to, the SVC or the IVC. The expandable anchoring mechanism 38 may also have an expanded configuration that locates the IMD 20 within the vasculature and secures the IMD 20 in place, with the cathode electrode 36 in engagement with the vasculature wall. In some cases, the expandable anchoring mechanism 38 may be configured to anchor the IMD 20 in the vasculature such that the length dimension D1 of the housing 22 is positioned parallel or substantially parallel (within 20 degrees of parallel) with blood flow through the vasculature. In some cases, the expandable anchoring mechanism 38 may resemble or be a stent, such as a braided stent, a woven stent or a laser cut stent. The expandable anchoring mechanism 38 may be self-expanding or could be balloon-expandable. It is contemplated that the expandable anchoring mechanism 38 may be formed of any desired metallic or polymeric material, as desired.

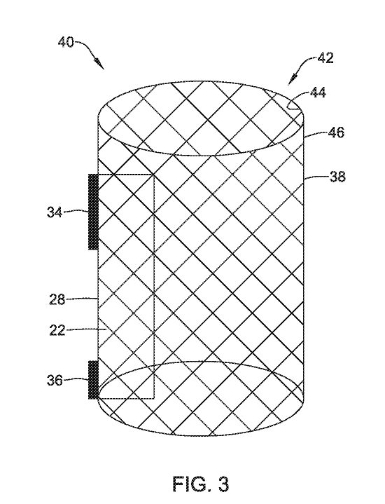

As noted above, the housing 22 may be disposed inside or outside of the expandable anchoring mechanism 38. FIG. 3 illustrates an illustrative IMD 40 in which the housing 22 is disposed inside the expandable anchoring mechanism 38. In some cases, the expandable anchoring mechanism 38 may be considered as having a side wall 42 that defines an inner surface 44 and an outer surface 46. In some cases, as shown in FIG. 3, the housing 22 may be secured relative to the inner surface 44. In some cases, the anode electrode 34 and/or the cathode electrode 36 may extend radially outwardly from the side wall 28 of the housing 22, in order to pass through the wall of the expandable anchoring mechanism 38 and to the vasculature wall to ensure good tissue contact.

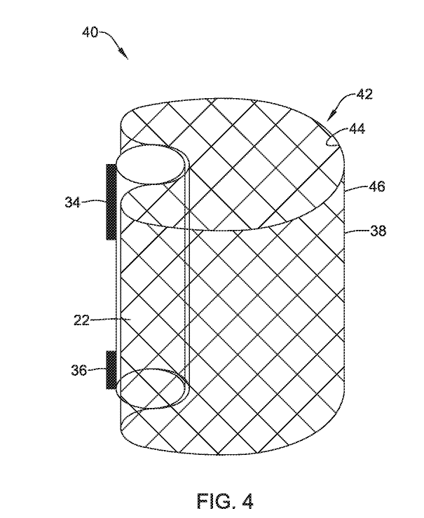

FIG. 4 shows an illustrative IMD 48 in which the housing 22 is secured relative to the outer surface 46 of the expandable anchoring mechanism 38. In some cases, this configuration may be useful in urging the anode electrode 34 and the cathode electrode 36 into good contact with the tissue in the vasculature. When so provided, the anode electrode 34 need not extend laterally out in the width dimension from the housing 22 as in FIG. 3.

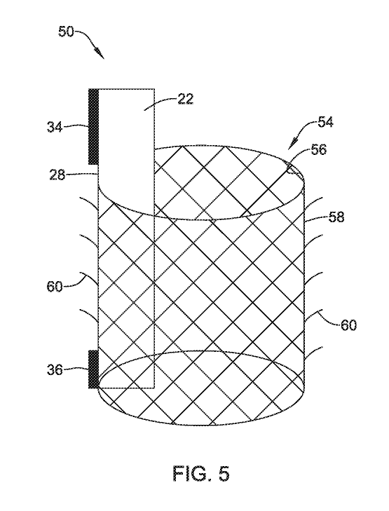

As shown for example in FIGS. 3 and 4, the expandable anchoring mechanism 38 may have an expanded or deployed configuration in which the housing 22 has a length that is roughly the same length as a length of the expandable anchoring mechanism 38. This is just one example. In some cases, the expandable anchoring mechanism 38 may instead have a deployed length that is greater than a length of the housing 22 (referenced as dimension D1 in FIG. 2). In some cases, the expandable anchoring mechanism 38 may have a deployed length that is less than a length of the housing 22. FIG. 5 provides an example of an IMD 50 having an expandable anchoring mechanism 52 that has a deployed length that is less than the length of the housing 22. In some cases, the expandable anchoring mechanism 52 includes a side wall 54 defining an inner surface 56 and an outer surface 58. As illustrated, the housing 22 in FIG. 5 is secured relative to the inner surface 56, but this is not required in all cases.

In some cases, the expandable anchoring mechanism 38 (or 52) may itself provide sufficient outward force on the vasculature to anchor the IMD in place within the vasculature. In some cases, the expandable anchoring mechanism 38 (or 52) may include fixation tines 60, shown extending radially outwardly from the outer surface 58. While the fixation tines 60 are illustrated as being part of the IMD 50, it will be appreciated that in some cases, the fixation tines 60 may be incorporated into other IMD's such as but not limited to those described herein.

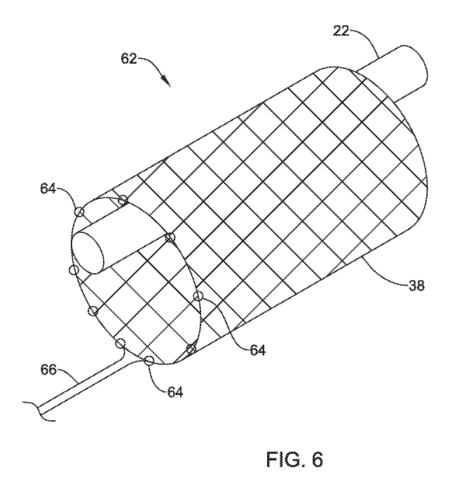

FIG. 6 is a schematic illustration of an IMD 62 in which the expandable anchoring mechanism 38 includes a plurality of anchor points or loops 64 that together accommodate a tether 66 that passes through each of the loops 64. The tether 66 extends away from the expandable anchoring mechanism 38 in a proximal direction. In some cases, during delivery of the IMD 62, there may be a desire to contract the expandable anchoring mechanism 38 from its expanded configuration (as illustrated) in order to reposition or replace the IMD 62 during the delivery and implantation process. By providing a proximal pull force on the tether 66, the tether 66 is able to contract the expandable anchoring mechanism 38 and thus permit removal or repositioning of the expandable anchoring mechanism 38 (and hence the IMD 62).

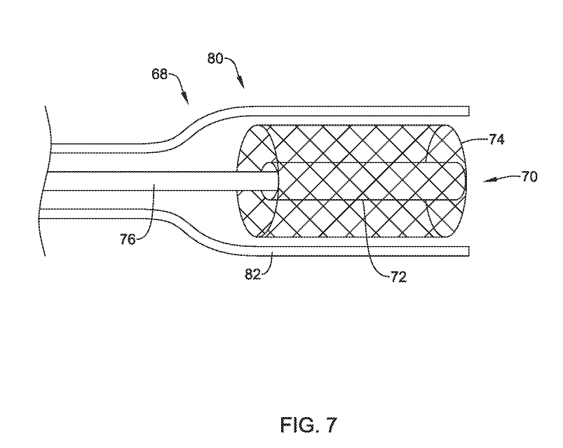

FIG. 7 provides an illustrative but non-limiting example of a delivery assembly 68 that may be used to deliver an IMD 70 including a housing 72 secured to an expandable anchoring mechanism 74. In some cases, particularly if the expandable anchoring mechanism 74 is self-expanding, the IMD 70 may be secured to a tubular member 76 that is itself disposed within the delivery assembly 68. In some cases, the delivery assembly 68 includes an outer tubular member 80 including a widened portion 82 that is configured to accommodate the IMD 70 therein. Once the delivery assembly 68 has been advanced to a desired location within the vasculature, such as but not limited to the SVC or the IVC (FIG. 1), the IMD 70 may be delivered by advancing the tubular member 76 distally to move the IMD 70 distally out of the widened portion 82 of the outer tubular member 80. In some cases, the tubular member 76 may be used to hold the IMD 70 in place while the outer tubular member 80 is withdrawn proximally. When the IMD 70 is pushed out the distal end of the widened portion 82 of the outer tubular member 80, the expandable anchoring mechanism 74 may self-expand from its lower profile collapsed or delivery configuration to its expanded anchoring configuration.

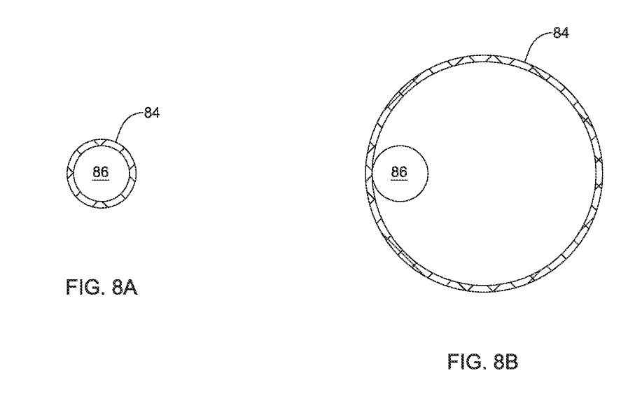

It will be appreciated that the collapsed or delivery configuration of the expandable anchoring mechanism 38, 74 may take a variety of different forms. In some cases, the expandable anchoring mechanism may simply expand from a compressed configuration to an expanded configuration, as shown for example in FIGS. 8A and 8B. In FIG. 8A, an expandable anchoring mechanism 84 may be seen as being compressed down onto a housing 86 (representative of the housing 22, for example). In FIG. 8B, it can be seen that the expandable anchoring mechanism 84 has expanded into its expanded configuration.

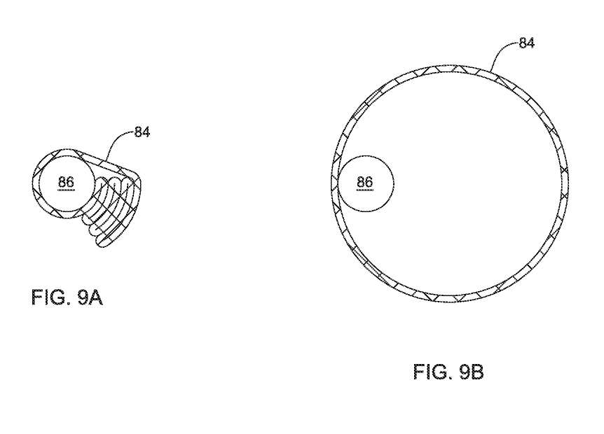

In some cases, the expandable anchoring mechanism 84 may be folded down into a delivery configuration, as shown in FIG. 9A. FIG. 9B then shows the expanded configuration of the expandable anchoring mechanism 84.

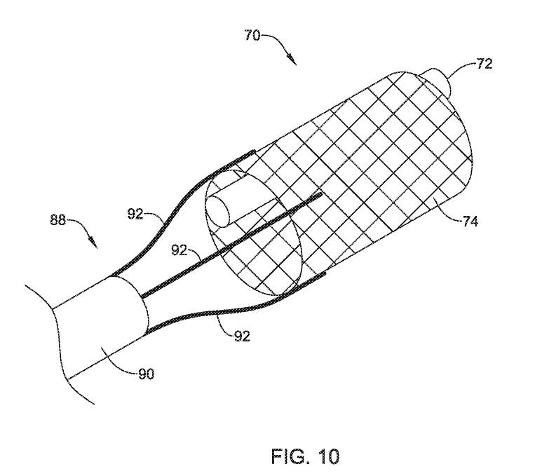

FIG. 10 illustrates another illustrative delivery system for the IMD 70. In FIG. 10, a delivery device 88 includes a tubular body 90 and several members 92 that extend from the tubular body 90. In some cases, the members 92 may be movable between a position in which the members 92 do not materially contact the IMD 70 and a position in which the members 92 sufficiently engage the IMD 70 to be able to push and/or pull the IMD 70 into a desired position. In some cases, an outer sheath (not shown) may extend over the tubular body 90 and the members 92 (and hence the IMD 70) while the IMD 70 is being advanced through the vasculature. The members 92 may simply provide a compressive force on the expandable anchoring mechanism 74 in order to engage the IMD 70. In some cases, the members 92 may include hooks or other structures (not shown) to facilitate engaging the IMD 70, or the expandable anchoring mechanism 74 itself may include features to help the members 92 engage the IMD 70.

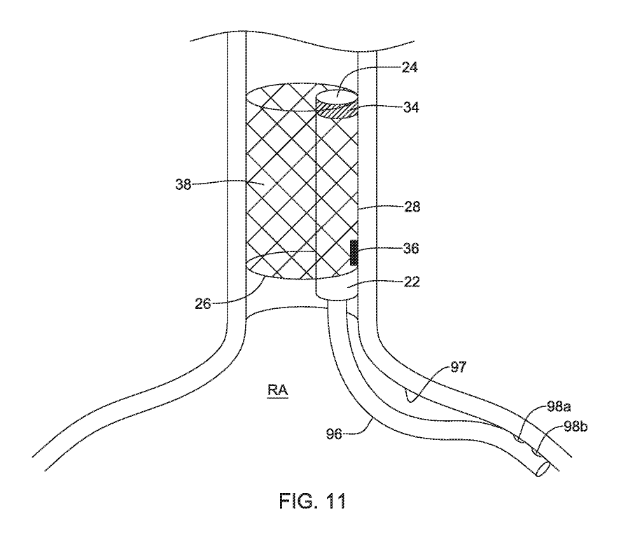

FIG. 11 is a schematic illustration of an IMD 94 implanted within the SVC. The IMD 94 is similar to the IMD 20 (FIG. 2), but includes a lead structure 96 extending from the second end 26 of the housing 22. In some cases, as illustrated, the anode electrode 34 may be a ring electrode located at or near the first end 24 of the housing 22. In the example shown, the lead structure 96 is configured to extend into the right atrium RA. While the lead structure 96 is shown extending into the right atrium RA, it will be appreciated that in some cases, the lead structure 96 may be configured to extend into the right ventricle RV. In some cases, the lead structure 96 may be configured to extend into cardiac vasculature such as but not limited to the coronary sinus.

In some cases, the lead structure 96 may have be biased into a curved shape that facilitates forcing the lead structure 96 into engagement with a wall 97 of the right atrium RA. In some cases, the lead structure 96 may include one or more electrodes, such as an electrode 98a and/or an electrode 98b. The electrodes 98a, 98b may be used in combination with the anode electrode 34 and the cathode electrode 36 for pacing within the right atrium RA. In some cases, the electrodes 98a, 98b, and others if present on the lead structure 96, may be used in place of the anode electrode 34 and/or the cathode electrode 36 for pacing within the right atrium RA. The electrodes 98a, 98b may be used in combination with or in place of the anode electrode 34 and/or the cathode electrode 36 to sense electrical activity in and/or near the right atrium RA. In some cases, the cathode electrode 36 may be omitted, and one or more of the electrodes 98a, 98b may be used as the cathode along with the anode electrode 34 to pace. In some cases, only a single electrode 98a may be provided on the lead structure 96.

In some cases, multiple spaced electrodes 98a, 98b may be provided along a length of the lead structure 96. Circuitry within housing 22 may be configured to select a particular electrode from the multiple spaced electrodes 98a, 98b for use as the cathode during subsequent pacing. In some cases, the circuitry may perform a capture threshold test and identify which of the multiple spaced electrodes 98a, 98b has the lowest capture threshold, and may then use that electrode during subsequent pacing of the right atrium.

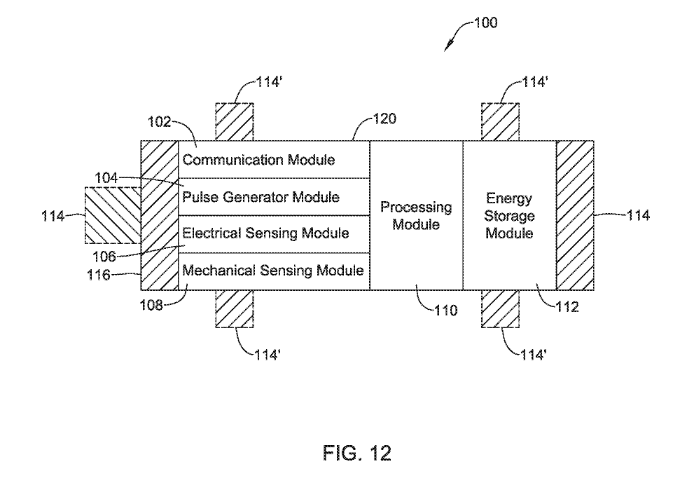

FIG. 12 is a conceptual schematic block diagram of an illustrative leadless cardiac pacemaker (LCP) that may be implanted on the heart or within a chamber of the heart and may operate to sense physiological signals and parameters and deliver one or more types of electrical stimulation therapy to the heart of the patient. Example electrical stimulation therapy may include bradycardia pacing, rate responsive pacing therapy, cardiac resynchronization therapy (CRT), anti-tachycardia pacing (ATP) therapy and/or the like. As can be seen in FIG. 12, the LCP 100 may be a compact device with all components housed within the LCP 100 or directly on a housing 120. In some instances, the LCP 100 may include one or more of a communication module 102, a pulse generator module 104, an electrical sensing module 106, a mechanical sensing module 108, a processing module 110, an energy storage module 112, and electrodes 114.

The LCP 100 may be considered as an example of the housing that forms part of the IMD 20 (FIG. 2), the IMD 40 (FIG. 3), the IMD 48 (FIG. 4), the IMD 50 (FIG. 5), the IMD 62 (FIG. 6), the IMD 70 (FIG. 7) and/or the IMD 94 (FIG. 11). It will be appreciated that particular features or elements described with respect to one of the IMD 20, the IMD 40, the IMD 48, the IMD 50, the IMD 62, the IMD 70 and/or the IMD 94 may be incorporated into any other of the IMD 20, the IMD 40, the IMD 48, the IMD 50, the IMD 62, the IMD 70 and/or the IMD 94.

As depicted in FIG. 12, the LCP 100 may include electrodes 114, which can be secured relative to the housing 120 and electrically exposed to tissue and/or blood surrounding the LCP 100. The electrodes 114 may generally conduct electrical signals to and from the LCP 100 and the surrounding tissue and/or blood. Such electrical signals can include communication signals, electrical stimulation pulses, and intrinsic cardiac electrical signals, to name a few. Intrinsic cardiac electrical signals may include electrical signals generated by the heart and may be represented by an electrocardiogram (ECG). The electrodes 114 may include one or more biocompatible conductive materials such as various metals or alloys that are known to be safe for implantation within a human body. In some instances, the electrodes 114 may be generally disposed on either end of the LCP 100 and may be in electrical communication with one or more of modules the 102, 104, 106, 108, and 110. In embodiments where the electrodes 114 are secured directly to the housing 120, an insulative material may electrically isolate the electrodes 114 from adjacent electrodes, the housing 120, and/or other parts of the LCP 100. In some instances, some or all of the electrodes 114 may be spaced from the housing 120 and may be connected to the housing 120 and/or other components of the LCP 100 through connecting wires. In such instances, the electrodes 114 may be placed on a tail (not shown) that extends out away from the housing 120.

As shown in FIG. 12, in some embodiments, the LCP 100 may include electrodes 114′. The electrodes 114′ may be in addition to the electrodes 114, or may replace one or more of the electrodes 114. The electrodes 114′ may be similar to the electrodes 114 except that the electrodes 114′ are disposed on the sides of the LCP 100. In some cases, the electrodes 114′ may increase the number of electrodes by which the LCP 100 may deliver communication signals and/or electrical stimulation pulses, and/or may sense intrinsic cardiac electrical signals, communication signals, and/or electrical stimulation pulses. While generically shown as being the same size, it will be appreciated that one of the electrodes 114′ may, for example, be relatively larger in surface area to be used as a pacing anode electrode while another of the electrodes 114′ may be relatively smaller in surface area to be used as a pacing cathode electrode.

The electrodes 114 and/or 114′ may assume any of a variety of sizes and/or shapes, and may be spaced at any of a variety of spacings. For example, the electrodes 114 may have an outer diameter of two to twenty millimeters (mm). In other embodiments, the electrodes 114 and/or 114′ may have a diameter of two, three, five, seven millimeters (mm), or any other suitable diameter, dimension and/or shape. Example lengths for the electrodes 114 and/or 114′ may include, for example, one, three, five, ten millimeters (mm), or any other suitable length. As used herein, the length is a dimension of the electrodes 114 and/or 114′ that extends away from the outer surface of the housing 120. In some cases, the housing includes a protrusion (not shown) that extends away from the side of the housing, where the protrusion carries an anode electrode (e.g. electrode 114 or 114′). The protrusion may help space the anode electrode away from the side of the housing and into engagement with the patient's vasculature. In some instances, at least some of the electrodes 114 and/or 114′ may be spaced from one another by a distance of twenty, thirty, forty, fifty millimeters (mm), or any other suitable spacing. The electrodes 114 and/or 114′ of a single device may have different sizes with respect to each other, and the spacing and/or lengths of the electrodes on the device may or may not be uniform.

In the illustrative embodiment shown, the communication module 102 may be electrically coupled to the electrodes 114 and/or 114′ and may be configured to deliver communication pulses to tissues of the patient for communicating with other devices such as sensors, programmers, other medical devices, and/or the like. Communication signals, as used herein, may be any modulated signal that conveys information to another device, either by itself or in conjunction with one or more other modulated signals. In some embodiments, communication signals may be limited to sub-threshold signals that do not result in capture of the heart yet still convey information. The communication signals may be delivered to another device that is located either external or internal to the patient's body. In some instances, the communication may take the form of distinct communication pulses separated by various amounts of time. In some of these cases, the timing between successive pulses may convey information. The communication module 102 may additionally be configured to sense for communication signals delivered by other devices, which may be located external or internal to the patient's body.

The communication module 102 may communicate to help accomplish one or more desired functions. Some example functions include delivering sensed data, using communicated data for determining occurrences of events such as arrhythmias, coordinating delivery of electrical stimulation therapy, and/or other functions. In some cases, the LCP 100 may use communication signals to communicate raw information, processed information, messages and/or commands, and/or other data. Raw information may include information such as sensed electrical signals (e.g. a sensed ECG), signals gathered from coupled sensors, and the like. In some embodiments, the processed information may include signals that have been filtered using one or more signal processing techniques. Processed information may also include parameters and/or events that are determined by the LCP 100 and/or another device, such as a determined heart rate, timing of determined heartbeats, timing of other determined events, determinations of threshold crossings, expirations of monitored time periods, accelerometer signals, activity level parameters, blood-oxygen parameters, blood pressure parameters, heart sound parameters, and the like. In some cases, processed information may, for example, be provided by a chemical sensor or an optically interfaced sensor. Messages and/or commands may include instructions or the like directing another device to take action, notifications of imminent actions of the sending device, requests for reading from the receiving device, requests for writing data to the receiving device, information messages, and/or other messages commands.

In at least some embodiments, the communication module 102 (or the LCP 100) may further include switching circuitry to selectively connect one or more of the electrodes 114 and/or 114′ to the communication module 102 in order to select which of the electrodes 114 and/or 114′ that the communication module 102 delivers communication pulses with. It is contemplated that the communication module 102 may be communicating with other devices via conducted signals, radio frequency (RF) signals, optical signals, acoustic signals, inductive coupling, and/or any other suitable communication methodology. Where the communication module 102 generates electrical communication signals, the communication module 102 may include one or more capacitor elements and/or other charge storage devices to aid in generating and delivering communication signals. In the embodiment shown, the communication module 102 may use energy stored in the energy storage module 112 to generate the communication signals. In at least some examples, the communication module 102 may include a switching circuit that is connected to the energy storage module 112 and, with the switching circuitry, may connect the energy storage module 112 to one or more of the electrodes 114/114′ to generate the communication signals.

As shown in FIG. 12, a pulse generator module 104 may be electrically connected to one or more of the electrodes 114 and/or 114′. The pulse generator module 104 may be configured to generate electrical stimulation pulses and deliver the electrical stimulation pulses to tissues of a patient via one or more of the electrodes 114 and/or 114′ in order to effectuate one or more electrical stimulation therapies. Electrical stimulation pulses as used herein are meant to encompass any electrical signals that may be delivered to tissue of a patient for purposes of treatment of any type of disease or abnormality. For example, when used to treat heart disease, the pulse generator module 104 may generate electrical stimulation pacing pulses for capturing the heart of the patient, i.e. causing the heart to contract in response to the delivered electrical stimulation pulse. In some of these cases, the LCP 100 may vary the rate at which the pulse generator module 104 generates the electrical stimulation pulses, for example in rate adaptive pacing. In other embodiments, the electrical stimulation pulses may include defibrillation/cardioversion pulses for shocking the heart out of fibrillation or into a normal heart rhythm. In yet other embodiments, the electrical stimulation pulses may include anti-tachycardia pacing (ATP) pulses. It should be understood that these are just some examples. When used to treat other ailments, the pulse generator module 104 may generate electrical stimulation pulses suitable for neurostimulation therapy or the like. The pulse generator module 104 may include one or more capacitor elements and/or other charge storage devices to aid in generating and delivering appropriate electrical stimulation pulses. In at least some embodiments, the pulse generator module 104 may use energy stored in the energy storage module 112 to generate the electrical stimulation pulses. In some particular embodiments, the pulse generator module 104 may include a switching circuit that is connected to the energy storage module 112 and may connect the energy storage module 112 to one or more of the electrodes 114/114′ to generate electrical stimulation pulses.

The LCP 100 may further include an electrical sensing module 106 and a mechanical sensing module 108. The electrical sensing module 106 may be configured to sense intrinsic cardiac electrical signals conducted from the electrodes 114 and/or 114′ to the electrical sensing module 106. For example, the electrical sensing module 106 may be electrically connected to one or more of the electrodes 114 and/or 114′ and the electrical sensing module 106 may be configured to receive cardiac electrical signals conducted through the electrodes 114 and/or 114′ via a sensor amplifier or the like. In some embodiments, the cardiac electrical signals may represent local information from the chamber in which the LCP 100 is implanted. For instance, if the LCP 100 is implanted within a ventricle of the heart, cardiac electrical signals sensed by the LCP 100 through the electrodes 114 and/or 114′ may represent ventricular cardiac electrical signals. The mechanical sensing module 108 may include, or be electrically connected to, various sensors, such as accelerometers, including multi-axis accelerometers such as two- or three-axis accelerometers, gyroscopes, including multi-axis gyroscopes such as two- or three-axis gyroscopes, blood pressure sensors, heart sound sensors, piezoelectric sensors, blood-oxygen sensors, and/or other sensors which measure one or more physiological parameters of the heart and/or patient. Mechanical sensing module 108, when present, may gather signals from the sensors indicative of the various physiological parameters. The electrical sensing module 106 and the mechanical sensing module 108 may both be connected to the processing module 110 and may provide signals representative of the sensed cardiac electrical signals and/or physiological signals to the processing module 110. Although described with respect to FIG. 12 as separate sensing modules, in some embodiments, the electrical sensing module 106 and the mechanical sensing module 108 may be combined into a single module. In at least some examples, the LCP 100 may only include one of the electrical sensing module 106 and the mechanical sensing module 108. In some cases, any combination of the processing module 110, the electrical sensing module 106, the mechanical sensing module 108, the communication module 102, the pulse generator module 104 and/or the energy storage module may be considered a controller of the LCP 100.

The processing module 110 may be configured to direct the operation of the LCP 100 and may, in some embodiments, be termed a controller. For example, the processing module 110 may be configured to receive cardiac electrical signals from the electrical sensing module 106 and/or physiological signals from the mechanical sensing module 108. Based on the received signals, the processing module 110 may determine, for example, occurrences and types of arrhythmias and other determinations such as whether the LCP 100 has become dislodged. The processing module 110 may further receive information from the communication module 102. In some embodiments, the processing module 110 may additionally use such received information to determine occurrences and types of arrhythmias and/or and other determinations such as whether the LCP 100 has become dislodged. In still some additional embodiments, the LCP 100 may use the received information instead of the signals received from the electrical sensing module 106 and/or the mechanical sensing module 108—for instance if the received information is deemed to be more accurate than the signals received from the electrical sensing module 106 and/or the mechanical sensing module 108 or if the electrical sensing module 106 and/or the mechanical sensing module 108 have been disabled or omitted from the LCP 100.

After determining an occurrence of an arrhythmia, the processing module 110 may control the pulse generator module 104 to generate electrical stimulation pulses in accordance with one or more electrical stimulation therapies to treat the determined arrhythmia. For example, the processing module 110 may control the pulse generator module 104 to generate pacing pulses with varying parameters and in different sequences to effectuate one or more electrical stimulation therapies. As one example, in controlling the pulse generator module 104 to deliver bradycardia pacing therapy, the processing module 110 may control the pulse generator module 104 to deliver pacing pulses designed to capture the heart of the patient at a regular interval to help prevent the heart of a patient from falling below a predetermined threshold. In some cases, the rate of pacing may be increased with an increased activity level of the patient (e.g. rate adaptive pacing). For instance, the processing module 110 may monitor one or more physiological parameters of the patient which may indicate a need for an increased heart rate (e.g. due to increased metabolic demand). The processing module 110 may then increase the rate at which the pulse generator module 104 generates electrical stimulation pulses. Adjusting the rate of delivery of the electrical stimulation pulses based on the one or more physiological parameters may extend the battery life of the LCP 100 by only requiring higher rates of delivery of electrical stimulation pulses when the physiological parameters indicate there is a need for increased cardiac output. Additionally, adjusting the rate of delivery of the electrical stimulation pulses may increase a comfort level of the patient by more closely matching the rate of delivery of electrical stimulation pulses with the cardiac output need of the patient.

For ATP therapy, the processing module 110 may control the pulse generator module 104 to deliver pacing pulses at a rate faster than an intrinsic heart rate of a patient in attempt to force the heart to beat in response to the delivered pacing pulses rather than in response to intrinsic cardiac electrical signals. Once the heart is following the pacing pulses, the processing module 110 may control the pulse generator module 104 to reduce the rate of delivered pacing pulses down to a safer level. In CRT, the processing module 110 may control the pulse generator module 104 to deliver pacing pulses in coordination with another device to cause the heart to contract more efficiently. In cases where the pulse generator module 104 is capable of generating defibrillation and/or cardioversion pulses for defibrillation/cardioversion therapy, the processing module 110 may control the pulse generator module 104 to generate such defibrillation and/or cardioversion pulses. In some cases, the processing module 110 may control the pulse generator module 104 to generate electrical stimulation pulses to provide electrical stimulation therapies different than those examples described above.

Aside from controlling the pulse generator module 104 to generate different types of electrical stimulation pulses and in different sequences, in some embodiments, the processing module 110 may also control the pulse generator module 104 to generate the various electrical stimulation pulses with varying pulse parameters. For example, each electrical stimulation pulse may have a pulse width and a pulse amplitude. The processing module 110 may control the pulse generator module 104 to generate the various electrical stimulation pulses with specific pulse widths and pulse amplitudes. For example, the processing module 110 may cause the pulse generator module 104 to adjust the pulse width and/or the pulse amplitude of electrical stimulation pulses if the electrical stimulation pulses are not effectively capturing the heart. Such control of the specific parameters of the various electrical stimulation pulses may help the LCP 100 provide more effective delivery of electrical stimulation therapy.

In some embodiments, the processing module 110 may further control the communication module 102 to send information to other devices. For example, the processing module 110 may control the communication module 102 to generate one or more communication signals for communicating with other devices of a system of devices. For instance, the processing module 110 may control the communication module 102 to generate communication signals in particular pulse sequences, where the specific sequences convey different information. The communication module 102 may also receive communication signals for potential action by the processing module 110.

In further embodiments, the processing module 110 may control switching circuitry by which the communication module 102 and the pulse generator module 104 deliver communication signals and/or electrical stimulation pulses to tissue of the patient. As described above, both the communication module 102 and the pulse generator module 104 may include circuitry for connecting one or more of the electrodes 114 and/or 114′ to the communication module 102 and/or the pulse generator module 104 so those modules may deliver the communication signals and electrical stimulation pulses to tissue of the patient. The specific combination of one or more electrodes by which the communication module 102 and/or the pulse generator module 104 deliver communication signals and electrical stimulation pulses may influence the reception of communication signals and/or the effectiveness of electrical stimulation pulses. Although it was described that each of the communication module 102 and the pulse generator module 104 may include switching circuitry, in some embodiments, the LCP 100 may have a single switching module connected to the communication module 102, the pulse generator module 104, and the electrodes 114 and/or 114′. In such embodiments, processing module 110 may control the switching module to connect the modules 102/104 and the electrodes 114/114′ as appropriate.

In some embodiments, the processing module 110 may include a pre-programmed chip, such as a very-large-scale integration (VLSI) chip or an application specific integrated circuit (ASIC). In such embodiments, the chip may be pre-programmed with control logic in order to control the operation of the LCP 100. By using a pre-programmed chip, the processing module 110 may use less power than other programmable circuits while able to maintain basic functionality, thereby potentially increasing the battery life of the LCP 100. In other instances, the processing module 110 may include a programmable microprocessor or the like. Such a programmable microprocessor may allow a user to adjust the control logic of the LCP 100 after manufacture, thereby allowing for greater flexibility of the LCP 100 than when using a pre-programmed chip. In still other embodiments, the processing module 110 may not be a single component. For example, the processing module 110 may include multiple components positioned at disparate locations within the LCP 100 in order to perform the various described functions. For example, certain functions may be performed in one component of the processing module 110, while other functions are performed in a separate component of the processing module 110.

The processing module 110, in additional embodiments, may include a memory circuit and the processing module 110 may store information on and read information from the memory circuit. In other embodiments, the LCP 100 may include a separate memory circuit (not shown) that is in communication with the processing module 110, such that the processing module 110 may read and write information to and from the separate memory circuit. The memory circuit, whether part of the processing module 110 or separate from the processing module 110, may be volatile memory, non-volatile memory, or a combination of volatile memory and non-volatile memory.

The energy storage module 112 may provide a power source to the LCP 100 for its operations. In some embodiments, the energy storage module 112 may be a non-rechargeable lithium-based battery. In other embodiments, the non-rechargeable battery may be made from other suitable materials. In some embodiments, the energy storage module 112 may be considered to be a rechargeable power supply, such as but not limited to, a rechargeable battery. In still other embodiments, the energy storage module 112 may include other types of energy storage devices such as capacitors or super capacitors. In some cases, as will be discussed, the energy storage module 112 may include a rechargeable primary battery and a non-rechargeable secondary battery. In some cases, the primary battery and the second battery, if present, may both be rechargeable.

The LCP 100 may be coupled to an expandable anchoring mechanism. The expandable anchoring mechanisms described herein, such as the expandable anchoring mechanism 38, may be made from a metal, metal alloy, polymer (some examples of which are disclosed below), a metal-polymer composite, ceramics, combinations thereof, and the like, or other suitable material. Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

As alluded to herein, within the family of commercially available nickel-titanium or nitinol alloys, is a category designated “linear elastic” or “non-super-elastic” which, although may be similar in chemistry to conventional shape memory and super elastic varieties, may exhibit distinct and useful mechanical properties. Linear elastic and/or non-super-elastic nitinol may be distinguished from super elastic nitinol in that the linear elastic and/or non-super-elastic nitinol does not display a substantial “superelastic plateau” or “flag region” in its stress/strain curve like super elastic nitinol does. Instead, in the linear elastic and/or non-super-elastic nitinol, as recoverable strain increases, the stress continues to increase in a substantially linear, or a somewhat, but not necessarily entirely linear relationship until plastic deformation begins or at least in a relationship that is more linear that the super elastic plateau and/or flag region that may be seen with super elastic nitinol. Thus, for the purposes of this disclosure linear elastic and/or non-super-elastic nitinol may also be termed “substantially” linear elastic and/or non-super-elastic nitinol.

In some cases, linear elastic and/or non-super-elastic nitinol may also be distinguishable from super elastic nitinol in that linear elastic and/or non-super-elastic nitinol may accept up to about 2-5% strain while remaining substantially elastic (e.g., before plastically deforming) whereas super elastic nitinol may accept up to about 8% strain before plastically deforming. Both of these materials can be distinguished from other linear elastic materials such as stainless steel (that can also can be distinguished based on its composition), which may accept only about 0.2 to 0.44 percent strain before plastically deforming.

In some embodiments, the linear elastic and/or non-super-elastic nickel-titanium alloy is an alloy that does not show any martensite/austenite phase changes that are detectable by differential scanning calorimetry (DSC) and dynamic metal thermal analysis (DMTA) analysis over a large temperature range. For example, in some embodiments, there may be no martensite/austenite phase changes detectable by DSC and DMTA analysis in the range of about −60 degrees Celsius (° C.) to about 120° C. in the linear elastic and/or non-super-elastic nickel-titanium alloy. The mechanical bending properties of such material may therefore be generally inert to the effect of temperature over this very broad range of temperature. In some embodiments, the mechanical bending properties of the linear elastic and/or non-super-elastic nickel-titanium alloy at ambient or room temperature are substantially the same as the mechanical properties at body temperature, for example, in that they do not display a super-elastic plateau and/or flag region. In other words, across a broad temperature range, the linear elastic and/or non-super-elastic nickel-titanium alloy maintains its linear elastic and/or non-super-elastic characteristics and/or properties.

In some embodiments, the linear elastic and/or non-super-elastic nickel-titanium alloy may be in the range of about 50 to about 60 weight percent nickel, with the remainder being essentially titanium. In some embodiments, the composition is in the range of about 54 to about 57 weight percent nickel. One example of a suitable nickel-titanium alloy is FHP-NT alloy commercially available from Furukawa Techno Material Co. of Kanagawa, Japan. Some examples of nickel titanium alloys are disclosed in U.S. Pat. Nos. 5,238,004 and 6,508,803, which are incorporated herein by reference. Other suitable materials may include ULTANIUM™ (available from Neo-Metrics) and GUM METAL™ (available from Toyota). In some other embodiments, a superelastic alloy, for example a superelastic nitinol can be used to achieve desired properties.

In some cases, an expandable anchoring mechanism such as the expandable anchoring mechanism 38 may be formed of, coated with or otherwise include one or more polymeric materials. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), Marlex high-density polyethylene, Marlex low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments.

What is claimed is:

1. A leadless cardiac pacemaker (LCP) configured for deployment within a patient's vasculature at a location near the patient's heart, the LCP comprising:
an elongated housing configured to be positioned within the patient's vasculature proximate the patient's heart, the elongated housing having opposing ends and a side wall extending between the opposing ends, the elongated housing having a length dimension between the opposing ends and a width dimension normal to the length dimension, wherein the length dimension is larger than the width dimension;
a power source disposed within the elongated housing;
circuitry disposed within the elongated housing and operatively coupled to the power source, the circuitry configured to pace the patient's heart and/or sense electrical activity of the patient's heart;
an anode electrode fixed relative to the elongated housing;
a cathode electrode fixed relative to the elongated housing, the cathode electrode spaced from the anode electrode and positioned along the side wall of the elongated housing, wherein the cathode electrode has a surface area that is smaller than a surface area of the anode electrode;
the anode electrode and the cathode electrode are operatively coupled to the circuitry; and
an expandable anchoring mechanism secured to the elongated housing, the expandable anchoring mechanism having a collapsed configuration for delivery and an expanded configuration that locates the LCP within the patient's vasculature with the cathode electrode in engagement with the patient's vasculature.

2. The LCP of claim 1, wherein the expandable anchoring mechanism is configured to anchor the LCP in the patient's vasculature such that the length dimension of the elongated housing is positioned substantially parallel with blood flow in the patient's vasculature.

3. The LCP of claim 1, wherein the anode electrode is disposed proximate a first opposing end of the elongated housing.

4. The LCP of claim 3, wherein the cathode electrode is disposed proximate a second opposing end of the elongated housing.

5. The LCP of claim 1, further comprising a retrieval feature disposed proximate at least one of the opposing ends of the elongated housing.

6. The LCP of claim 1, wherein the expandable anchoring mechanism comprises a side wall defining an inner surface and an outer surface, the elongated housing is secured to the inner surface of the expandable anchoring mechanism with the cathode electrode extending laterally outwardly from the elongated housing in the width dimension and through the side wall.

7. The LCP of claim 1, wherein the expandable anchoring mechanism comprises a side wall defining an inner surface and an outer surface, the elongated housing is secured to the outer surface of the expandable anchoring mechanism with the cathode electrode held in engagement with the patient's vasculature.

8. The LCP of claim 1, further comprising a lead structure extending from the elongated housing, the lead structure including at least one additional electrode that is operatively coupled to the circuitry and configured to extend into the patient's heart from the patient's vasculature at the location near the patient's heart.

9. The LCP of claim 1, wherein the expandable anchoring mechanism is configured to anchor the LCP in the patient's superior vena cava proximate the patient's right atrium.

10. The LCP of claim 1, wherein the expandable anchoring mechanism is configured to anchor the LCP in the patient's inferior vena cava proximate the patient's right atrium.

11. The LCP of claim 1, further comprising a tether woven into an end of the expandable anchoring mechanism, the tether enabling the expandable anchoring mechanism to be at least partially collapsed from its expanded configuration for repositioning of the LCP.

12. The LCP of claim 1, wherein the expandable anchoring mechanism is configured such that in its expanded configuration, the expandable anchoring mechanism exerts sufficient outward force on the patient's vasculature to secure the LCP in place with the cathode electrode in engagement with the patient's vasculature.

13. The LCP of claim 1, wherein the expandable anchoring mechanism comprises a side wall defining an inner surface and an outer surface, and wherein the expandable anchoring mechanism further comprises fixation tines that extend outwardly from the outer surface of the side wall.

14. The LCP of claim 1, wherein the expandable anchoring mechanism has a length in its expanded configuration that is less than the length dimension of the elongated housing.

15. An implantable medical device (IMD) configured for deployment within a patient's vena cava, proximate the patient's right atrium, in order to pace the right atrium and/or sense electrical activity within the right atrium, the IMD comprising:
a housing configured to be positioned within the patient's vena cava, the housing having a first end and an opposing second end with a side wall extending between the first end and the opposing second end;
a power source disposed within the housing;
circuitry disposed within the housing and operatively coupled to the power source;
an anode electrode positioned proximate the first end of the housing;
a cathode electrode spaced from the first end of the housing, wherein the cathode electrode has a surface area that is smaller than a surface area of the anode electrode;
the anode electrode and the cathode electrode are operatively coupled to the circuitry; and
an expandable anchoring mechanism secured to the housing, the expandable anchoring mechanism having a collapsed configuration for delivery and an expanded configuration that locates the IMD within the vena cava with the cathode electrode in engagement with the vena cava.

16. The IMD of claim 15, further comprising a retrieval feature disposed proximate the first end of the housing.

17. The IMD of claim 15, further comprising a lead that comprises at least one additional electrode that is operatively coupled to the circuitry, the lead is configured to extend into the patient's heart.

18. The IMD of claim 15, where the lead is configured to bias the at least one additional electrode against a wall of the right atrium.

19. A leadless cardiac pacemaker (LCP) configured for deployment within a patient's vasculature at a location near the patient's heart, the LCP comprising:
a housing configured to be positioned within the patient's vasculature proximate the patient's heart;
a power source disposed within the housing;
circuitry disposed within the housing and operatively coupled to the power source;
a lead comprising at least one electrode that is operatively coupled to the circuitry, the lead is configured to extend into the patient's heart;
the circuitry is configured to pace the patient's heart and/or sense electrical activity of the patient's heart using the at least one electrode of the lead; and
an expandable anchoring mechanism having a delivery configuration and an anchoring configuration, where the anchoring configuration anchors the LCP within the patient's vasculature at the location near the patient's heart.

20. The LCP of claim 19, wherein the expandable anchoring mechanism is configured to anchor the LCP within the superior vena cava or the inferior vena cava, and the lead is configured to bias the at least one electrode against a wall of the right atrium of the patient's heart.
